SEQUENCE LISTING (45 sequences)

SEQ ID NO: 1 - DNA, 93 bp, Homo sapiens
  misc_feature (1)..(93): Human proinsulin C-peptide CDS (Genbank NM_000207.2..gi109148525)
gaggcagaggacctgcaggtggggcaggtggagctgggcgggggccctggtgcaggcagcctgcagcccttggccctggaggggtccctgcag

SEQ ID NO: 2 - protein, 31 aa, Hominidae sp.
  MISC_FEATURE (1)..(31): Hominidae sp. proinsulin C-peptide  Human (Genbank P01308..gi124617), Chimpanzee (Genbank NP_001008996.1.. gi57113877), Orangutan (Genbank Q8HXV2..gi48428254), and Gorilla (Genbank AAM76640.1..gi23379796)
EAEDLQVGQVELGGGPGAGSLQPLALEGSLQ

SEQ ID NO: 3 - protein, 31 aa, Catarrhini sp.
  MISC_FEATURE (1)..(31): Catarrhini sp. proinsulin C-peptide  Rhesus macaque (Genbank 1006230A..gi223965), Crab-eating macaque (Genbank P30406..gi266373), and Green monkey (Genbank P30407..gi266362)
EAEDPQVGQVELGGGPGAGSLQPLALEGSLQ

SEQ ID NO: 4 - protein, 29 aa, Aotus trivirgatus
  MISC_FEATURE (1)..(29): Platyrrhini sp. proinsulin C-peptide  Northern night monkey (Genbank P67972..gi54037402)
EAEDLQVGQVELGGGSITGSLPPLEGPMQ

SEQ ID NO: 5 - protein, 26 aa, Bovidae-Caprinae sp.
  MISC_FEATURE (1)..(26): Bovidae-Caprinae sp. proinsulin C-peptide Cattle (Genbank P01317..gi124564), Water buffalo (Genbank BAE80772.1..gi89331178), European bison (Genbank 1312309A..gi225748), and Sheep (Genbank P01318..gi1708500)
EVEGPQVGALELAGGPGAGGLEGPPQ

SEQ ID NO: 6 - protein, 31 aa, Equidae sp.
  MISC_FEATURE (1)..(31): Equidae sp. proinsulin C-peptide  Horse (Genbank P01310..gi124616) and Przewalskii horse (Genbank AAB25818.1..gi299301)
EAEDPQVGEVELGGGPGLGGLQPLALAGPQQ

SEQ ID NO: 7 - protein, 29 aa, Sus scrofa
  MISC_FEATURE (1)..(29): Suidae sp. proinsulin C-peptide Sus scrofa (Genbank P01315..gi12643972)
EAENPQAGAVELGGGLGGLQALALEGPPQ

SEQ ID NO: 8 - DNA, 93 bp, Felis catus
  misc_feature (1)..(93): Felis catus proinsulin C-peptide CDS (Genbank AB043535.1..gi18376636)
gaggcggaggacctccaggggaaggacgcggagctgggggaggcgcctggcgccggcggcctgcagccctcggccctggaggcgcccctgcag

SEQ ID NO: 9 - protein, 31 aa, Felis catus
  MISC_FEATURE (1)..(31): Felidae sp. proinsulin C-peptide  Felis catus (Genbank P06306..gi90110828)
EAEDLQGKDAELGEAPGAGGLQPSALEAPLQ

SEQ ID NO: 10 - DNA, 93 bp, Canis familiaris
  misc_feature (1)..(93): Canis familiaris proinsulin C-peptide CDS (Genbank V00179.1..gi994)
gaggtggaggacctgcaggtgagggacgtggagctggccggggcgcctggcgagggcggcctgcagcccctggccctggagggggccctgcag

SEQ ID NO: 11 - protein, 31 aa, Canis familiaris
  MISC_FEATURE (1)..(31): Canidae sp. proinsulin C-peptide  Canis familiaris (Genbank P01321..gi124575)
EVEDLQVRDVELAGAPGEGGLQPLALEGALQ

SEQ ID NO: 12 - protein, 31 aa, Oryctolagus cuniculus
  MISC_FEATURE (1)..(31): Lagomorpha sp. proinsulin C-peptide Oryctolagus cuniculus (Genbank P01311..gi1352467)
EVEELQVGQAELGGGPGAGGLQPSALELALQ

SEQ ID NO: 13 - protein, 29 aa, Mus sp.
  MISC_FEATURE (1)..(29): Mouse sp. proinsulin type 1 C-peptide  House mouse (Genbank P01325..gi124511) and Western wild mouse (Genbank AAB60473.1..gi497063)
EVEDPQVEQLELGGSPGDLQTLALEVARQ

SEQ ID NO: 14 - protein, 31 aa, Mus sp.
  MISC_FEATURE (1)..(31): Mouse sp. proinsulin type 2 C-peptide  House mouse (Genbank P01326..gi124519), Western wild mouse (Genbank CAJ76270.1..gi86989480, and Ryukyu mouse (Genbank ABB89749.1..gi82749730)
EVEDPQVAQLELGGGPGAGDLQTLALEVAQQ

SEQ ID NO: 15 - protein, 29 aa, Mus caroli
  MISC_FEATURE (1)..(29): Ryukyu mouse proinsulin type 1 C-peptide  Mus caroli (Genbank ABB89745.1..gi82749722)
EVEDPQVEQLELGGSPGDLQTLALEVARE

SEQ ID NO: 16 - protein, 31 aa, Apodemus semotus
  MISC_FEATURE (1)..(31): Taiwan field mouse proinsulin type 1 C-peptide Apodemus semotus (Genbank ABB89744.1..gi82749720)
EVEDPQVEQLELGGAPGTGDLETLALEVARQ

SEQ ID NO: 17 - protein, 31 aa, Rattus norvegicus
  MISC_FEATURE (1)..(31): Norway rat proinsulin type 1 C-peptide (Genbank AAA41442.1..gi204957)
EVEDPQVPQLELGGGPEAGDLQTLALEVARQ

SEQ ID NO: 18 - protein, 31 aa, Rattus norvegicus
  MISC_FEATURE (1)..(31): Rat sp. proinsulin type 2 C-peptiide  Rattus norvegicus (Genbank AAA41443.1..gi204959) and Apodemus semotus (Genbank ABB89748.1..gi82749728)
EVEDPQVAQLELGGGPGAGDLQTLALEVARQ

SEQ ID NO: 19 - protein, 31 aa, Rattus losea
  MISC_FEATURE (1)..(31): Lesser rice-field rat proinsulin type 1 C-peptide (Genbank ABB89743.1..gi82749718)
EVEDPQVPQLELGGSPEAGDLQTLALEVARQ

SEQ ID NO: 20 - protein, 31 aa, Rattus losea
  MISC_FEATURE (1)..(31): Lesser rice-field rat proinsulin type 2 C-peptide (Genbank ABB89747.1..gi82749726)
EVEDPQVAQQELGGGPGAGDLQTLALEVARQ

SEQ ID NO: 21 - protein, 31 aa, Niviventer coxingi
  MISC_FEATURE (1)..(31): Coxing′s white-bellied rat proinsulin type 1 C-peptide (Genbank ABB89746.1..gi82749724)
EVEDPQVAQLELGEGPEAGDLQTLALEVARQ

SEQ ID NO: 22 - protein, 31 aa, Niviventer coxingi
  MISC_FEATURE (1)..(31): Coxing′s white-bellied rat proinsulin type 2 C-peptide (Genbank ABB89750.1..gi82749732)
EVEDPQVPQLELGGGPGTGDLQTLALEVARQ

SEQ ID NO: 23 - protein, 31 aa, Rodentia sp.
  MISC_FEATURE (1)..(31): Rodentia sp. proinsulin C-peptide (Genbank P21563..gi124615)
EVEDPQVGQVELGAGPGAGSEQTLALEVARQ

SEQ ID NO: 24 - protein, 31 aa, Spermophilus tridecemlineatus
  MISC_FEATURE (1)..(13): Thirteen-lined ground squirrel proinsulin C-peptide (Genbank Q91XI3..gi29427536)
EVEEQQGGQVELGGGPGAGLPQPLALEMALQ

SEQ ID NO: 25 - protein, 31 aa, Meriones unguiculatus
  MISC_FEATURE (1)..(31): Mongolian gerbil proinsulin C-peptide (Genbank ABB89751.1..gi82749734)
GVEDPQMPQLELGGSPGAGDLQALALEVARQ

SEQ ID NO: 26 - protein, 31 aa, Psammomys obesus
  MISC_FEATURE (1)..(31): Fat sand rat proinsulin C-peptide (Genbank Q62587..gi2497407)
GVDDPQMPQLELGGSPGAGDLRALALEVARQ

SEQ ID NO: 27 - protein, 31 aa, Microtus kikuchii
  MISC_FEATURE (1)..(31): Taiwan vole proinsulin C-peptide (Genbank ABB89752.1..gi82749736)
GVEDPQVAQLELGGGPGAGDLQTLALEVAQQ

SEQ ID NO: 28 - protein, 31 aa, Cricetulus longicaudatus
  MISC_FEATURE (1)..(31): Long-tailed hamster proinsulin C-peptide (Genbank P01313..gi124588)
GVEDPQVAQLELGGGPGADDLQTLALEVAQQ

SEQ ID NO: 29 - protein, 31 aa, Cavia porcellus
  MISC_FEATURE (1)..(31): Cavy proinsulin type 1 C-peptide (Genbank P01329..gi124581)
ELEDPQVEQTELGMGLGAGGLQPLALEMALQ

SEQ ID NO: 30 - protein, 29 aa, Cavia porcellus
  MISC_FEATURE (1)..(29): Cavy proinsulin type 2 C-peptide (Genbank)740063A..gi229473
ELEDPQVEQTELGMGLGAGGLQPLQGALQ

SEQ ID NO: 31 - protein, 31 aa, Chinchilla brevicaudata
  MISC_FEATURE (1)..(31): Chinchilla proinsulin C-peptide (Genbank P01327..gi83303940)
ELEDPQVGQADPGVVPEAGRLQPLALEMTLQ

SEQ ID NO: 32 - protein, 29 aa, Octodon degus
  MISC_FEATURE (1)..(29): Chilean brush tail rat proinsulin C-peptide (Genbank P17715..gi124651)
ELEDLQVEQAELGLEAGGLQPSALEMILQ

SEQ ID NO: 33 - protein, 28 aa, Gallus gallus
  MISC_FEATURE (1)..(29): Chicken and Pigeon proinsulin C-peptide Gallus gallus (Genbank P67970..gi54037398) and Columba livia (Genbank AAP45955.1..gi32891835 and AAP45983.1..gi32891891)
DVEQPLVSSPLRGEAGVLPFQQEEYEKV

SEQ ID NO: 34 - protein, 28 aa, Meleagris gallopavo
  MISC_FEATURE (1)..(28): Turkey proinsulin C-peptide (Genbank AAP45962.1..gi32891849 and AAP45990.1..gi32891905)
DVEQPLVGSPLRGEAGVLPFQQEEYEKV

SEQ ID NO: 35 - protein, 28 aa, Anas sp.
  MISC_FEATURE (1)..(28): Duck proinsulin C-peptide A. platyrhynchos var. Mallard (Genbank P01333..gi124547); A. platyrhynchos var. Peking (Genbank AAP45950.1..gi32891825 & AAP45978.1..gi32891881); A. crecca (Genbank AAP45949.1..gi32891823 & AAP45977.1..gi32891879)
DVEQPLVNGPLHGEVGELPFQHEEYQKV

SEQ ID NO: 36 - protein, 28 aa, Xenopus laevis
  MISC_FEATURE (1)..(28): African clawed frog proinsulin type 1 C-peptide (Genbank P12706..gi124513)
DMEQALVSGPQDNELDGMQLQPQEYQKM

SEQ ID NO: 37 - protein, 28 aa, Xenopus laevis
  MISC_FEATURE (1)..(28): African clawed frog proinsulin type 2 C-peptide (Genbank P12707..gi124526)
DIEQAQVNGPQDNELDGMQFQPQEYQKM

SEQ ID NO: 38 - protein, 5 aa, Artificial
  C-peptide residues 27-31
EGSLQ

SEQ ID NO: 39 - protein, 12 aa, Artificial
  C-peptide residues 20-31 from human sequence
SLQPLALEGSLQ

SEQ ID NO: 40 - protein, 22 aa, Artificial
  C22 C-peptide residues 10-31 from human sequence
VELGGGPGAGSLQPLALEGSLQ

SEQ ID NO: 41 - protein, 9 aa, Artificial
  Cmid C-peptide residues 11-19
ELGGGPGAG

SEQ ID NO: 42 - protein, 13 aa, Artificial
  N13 C-peptide residues 1-13
EAEDLQVGQVELG

SEQ ID NO: 43 - protein, 5 aa, Artificial
  AGSLQ C-peptide residues 27-31 (E27A)
AGSLQ

SEQ ID NO: 44 - protein, 5 aa, Artificial
  DGSLQ C-peptide residues 27-31 (E27D)
DGSLQ

SEQ ID NO: 45 - protein, 5 aa, Artificial
  SQLEG C-peptide residues 27-31 (scrambled)
SQLEG

(19) United States
(12) Patent Application Publication
(10) Pub. No.: US 2011/0020849 A1
(43) Pub. Date: Jan. 27, 2011

(54) BIOMARKERS FOR DIAGNOSTIC AND THERAPEUTIC METHODS

(75) Inventor: Dana Spence, Howell, MI (US)

Correspondence Address: HARNESS, DICKEY & PIERCE, P.L.C., P.O. BOX 828, BLOOMFIELD HILLS, MI, 48303, US

(73) Assignee: Wayne State University, Detroit, MI (US)

(21) Appl. No.: 12/565,570

(22) Filed: Sep. 23, 2009

Related U.S. Application Data
(63) Continuation of application No. PCT/US2008/003809, filed on Mar. 22, 2008.
(60) Provisional application No. 60/919,956, filed on Mar. 23, 2007.

(51) Int. Cl. C12Q 1/66; C12Q 1/02; C12Q 1/54; C12M 1/34

(52) U.S. Cl. 435/8; 435/29; 435/14; 435/287.3

(57) ABSTRACT

Erythrocyte ATP-release modulators and composition and methods for their use as biomarkers of glucose processing or vascular disorders, as well as methods for screening to identify to modulators; methods for monitoring efficacy of therapy; and apparatus for use in such methods.

### CROSS-REFERENCE TO RELATED APPLICATIONS

This application is a continuation of International Application No. PCT/US2008/003809, filed Mar. 22, 2008, which claims the benefit of U.S. Provisional Application No. 60/919,956, filed Mar. 23, 2007. The entire disclosures of the above applications are incorporated herein by reference.

### GOVERNMENT RIGHTS

This invention was made with Governmental Support by NIH Grant No. HL 073942 awarded by the National Institutes of Health. The Government has certain rights in this invention.

### BACKGROUND

The present disclosure relates to methods and apparatus using erythrocyte ATP release as a biomarker for disease detection, drug development, and therapeutic efficacy testing, which are useful in regard to glucose processing disorders and other erythrocyte-membrane-altering pathological conditions.

A growing prevalence of glucose processing disorders has resulted in a drive to develop new or improved treatments. As a result the pharmaceutical industry and academic research community are investing millions of dollars to provide advanced therapies for such conditions. Examples of these disorders include diabetes, e.g., diabetes mellitus types 1 and 2, gestational diabetes; and metabolic syndrome or its symptoms such as glucose intolerance, insulin insensitivity, and hyperglycemia.

While a number of tests exist to help diagnose diabetes, e.g., fasting blood glucose levels, tests for developing conditions, such as a metabolic syndrome preliminary to diabetes mellitus type 2, are still being developed. As a result, it would be beneficial to provide new types of tests that can be used, either alone or jointly with other tests, to provide a basis for diagnosis of such glucose processing disorders.

Moreover, because treatments for glucose processing disorders are undergoing intensive research, a number of different assays are being practiced to identify candidate pharmaceuticals and/or to assess their degree of efficacy. For example, insulin receptor ligand-binding or antibody-binding assays, calcineurin- or NFAT-activation assays, insulin biosynthesis-controlling receptor ligand assays (e.g., for incretin receptor binding), nuclear hormone receptor binding assays (e.g., for PPAR binding), and in vivo glucose assays are in use. Yet, these assays typically require either pre- or post-reaction of the test reagents or analytes, or isotopic labeling, to permit a detectable signal to be obtained as the assay result. Thus, it would be beneficial to provide new types of tests, offering greater ease of use, that can be employed as alternatives to or supplements for existing drug screening and efficacy tests in the area of medical treatments affecting glucose uptake. It would further be advantageous to provide new biomarkers that can function as diagnostic indicators and/or as drug identification or efficacy test targets.

### SUMMARY

The present technology provides assays and apparatus that permit detection of glucose processing disorders and candidate drug screening and efficacy testing of glucose processing disorder therapies. These assays are based on the use of erythrocyte ATP release as a novel biomarker. In various embodiments, the present technology further provides methods for assessing the health status of a human or other animal subject, comprising performing an ATP release assay on erythrocytes of said subject to obtain an ATP release assay level, and comparing said assay level to a reference level of ATP release. Preferably, said reference level is a normal range of ATP release determined by assaying erythrocytes of normal subjects under conditions substantially identical to said assaying of erythrocytes of said subject. In various embodiments, the assaying comprises obtaining a suspension of said erythrocytes, applying said physical force to said suspension so as to deform said erythrocytes, and detecting ATP levels in said suspension. For example, the method may comprise obtaining a suspension of erythrocytes, admixing the erythrocytes with luciferin to form a suspension having a pH 6.5 to about pH 8, contacting the suspension with luciferase, and observing the suspension for the presence of luciferase-catalyzed luminescence.

Also provided are methods for determining the efficacy of erythrocyte ATP-release activity of a compound, comprising
(A) contacting a sample of erythrocytes with said compound to prepare treated erythrocytes;
(B) assaying said treated erythrocytes for their level of ATP release to obtain a treated erythrocyte ATP release assay level; and
(C) comparing said treated erythrocyte ATP release assay level with control erythrocyte ATP release assay level, so as to determine the relative efficacy of said compound.
   In some embodiments, methods comprise screening substances to identify a candidate erythrocyte ATP-release modulator, wherein the methods comprise:
(A) providing a test substance and a sample of erythrocytes
(B) contacting a first portion of said sample of erythrocytes with said substance to prepare treated erythrocytes;
(C) assaying said treated erythrocytes for their level of ATP release to obtain a treated erythrocyte ATP release assay level;
(D) assaying a second portion of said sample of erythrocytes to obtain a control erythrocyte ATP release assay level; and
(E) comparing said treated erythrocyte ATP release assay level with said control erythrocyte ATP release assay level.
   Also provided are apparatus for measuring the level of erythrocyte ATP release by erythrocytes, comprising:
(A) a fluid flow conduit having a first region having a first cross-sectional area and an adjacent second region having a cross-sectional area that is less than said first cross-sectional area;
(B) a biocompatible pump in fluid communication with said fluid flow channel, operable to pump fluid from said first region to said second region; and
(C) a photodetector in optical communication with said second region of said fluid flow channel.

### FIGURES

The drawings described herein are for illustration purposes only and are not intended to limit the scope of the present disclosure in any way.

FIG. 1 presents bar chart results of determination of ATP release from rabbit RBCs subjected to flow in the presence and absence of a freshly prepared C-peptide preparation.

FIG. 2 presents bar chart results of determination of ATP release from diabetic human RBCs: 63.6±12.6 nM ATP release at 0 hours; and 256.1±38.7 nM ATP release after 6 hours of incubation with a freshly prepared C-peptide preparation. Error bars are ±SEM (n=7).

FIGS. 3A and 3B present electrospray ionization mass spectrograms (ESI-MS) of a freshly made C-peptide preparation (3A) and a C-peptide preparation after refrigeration for 24 hours (3B).

FIGS. 4A and 4B present an ESI-MS of C-peptide incubated with iron II (4A); and a chart (4B) of results of ATP release by rabbit RBCs incubated with iron II (390.6±6.3 nM) and with iron II/C-peptide (1000±23.0 nM). Error bars are ±SEM.

FIGS. 5A and 5B present an ESI-MS of C-peptide incubated with chromium III (5A); and a chart (5B) of result of ATP release by rabbit RBCs incubated with chromium III (303.6±13.0 nM) and with chromium III/C-peptide (743.7±54.1 nM).

FIG. 6 presents a chart of results of ATP release by human RBCs (537.3±7.2 nM) incubated with C-peptide and iron II (729.3±49.7 nM) or with C-peptide and chromium III (1292±61.4 nM) after 72 hours.

FIG. 7 presents a schematic drawing of an embodiment of an apparatus useful for detecting and/or measuring RBC ATP release.

FIG. 8 presents a number of exemplary rotary “chip” designs useful for detecting and/or measuring RBC ATP release.

FIG. 9 presents two multi-layer stationary “chip” design useful for detecting and/or measuring RBC ATP release.

It should be noted that the figures set forth herein are intended to exemplify the general characteristics of the compounds, compositions, and methods among those of this technology, for the purpose of the description of such embodiments herein. These figures may not precisely reflect the characteristics of any given embodiment, and are not necessarily intended to define or limit specific embodiments within the scope of this technology.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary,”) and sub-headings (such as “Assays”) used herein are intended only for general organization of topics within the disclosure of the technology, and are not intended to limit the disclosure of the technology or any aspect thereof. In particular, subject matter disclosed in the “Field” and “Background” may include aspects of technology within the scope of an invention, and may not constitute a recitation of prior art. Subject matter disclosed in the “Summary” is not an exhaustive or complete disclosure of the entire scope of the technology or any embodiments thereof.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

In various embodiments hereof, assays are provided for detecting and/or measuring erythrocyte ATP-release. Thus, erythrocyte ATP-release can be detected in order to assess the health status of a human or animal subject, or to screen for compounds that exhibit activity as erythrocyte ATP-release response modulators. In some embodiments, an assay hereof can be used to screen for the activity of compounds being developed for use as serum glucose clearance promoters or for use as vasodilation promoters.

### Assays

The present technology provides methods for comparative measurement of the ATP release by erythrocytes comprising performing an ATP release assay on a sample of erythrocytes to obtain an ATP release assay level, and comparing said assay level to a reference level of ATP release. In various embodiments, the reference level is a normal range of ATP release determined by assaying erythrocytes of normal subjects under conditions substantially identical to said assaying of erythrocytes of said subject.

In various embodiments, assay methods are performed on a suspension of erythrocytes. The suspension may consist of whole blood taken from a subject, or may be formed from isolated erythrocytes suspended in a biocompatible medium (i.e., a medium which in which the erythrocytes retain sufficient viability during the period of the assay so as to release ATP). Depending on the specific comparative measurement to be performed, the sample of erythrocytes may be obtained from any of a variety of sources. For example, the sample may be obtained from a human or other animal subject in methods for assessing the health status of the subject.

The assays of the present technology measure ATP release by erythrocytes. Such measurement may be performed by any suitable method, including methods among those known in the art. Methods useful herein include ATP-utilizing enzyme-catalyzed reactions, such as those using luciferin and luciferase.

In various embodiments, assays measure the release of ATP from erythrocytes following stimulation of the erythrocytes. Preferably, the stimulation is by application of physical force to the erythrocytes, in particular force sufficient to deform the erythrocyte in one or more dimensions. In some methods, however, in which ATP release is found to be quite pronounced, no such force may need to be applied in order to obtain a test result.

In some embodiments, a force is typically applied to a suspension comprising erythrocytes and luciferin, in contact with luciferase, in order to physically deform the plasma membrane of at least some of the erythrocytes. The physical deformation causes release of ATP from the erythrocytes, and the ATP diffuses to the luciferase, thereby permitting the enzyme to catalyze the conversion of luciferin substrate, generating bioluminescence. The reaction occurs in a chamber in or from which a photodetector can detect the light, either qualitatively or quantitatively. In various embodiments, the photodetector can be operatively attached to a recording device to record the detection results.

In some embodiments, the enzyme, e.g., luciferase, can be immobilized to the inner surface of the reaction chamber wall, or to beads, plates, or other solid surface(s) in contact with the erythrocyte cell suspension or with at least the aqueous medium thereof. For example immobilized enzyme electrodes can be used. Similarly a DNA Hybridization Chain Reaction can be used as described in R. M. Dirks & N. A. Pierce, in PNAS (USA) 101(43):15275-78 (Oct. 26, 2004) (e-Publ. Oct. 18, 2004; 10.1073/pnas.0407024101).

### Apparatus

The present technology provides an apparatus for an ATP release assay on erythrocytes. Such an apparatus may comprise a reaction chamber for use in a batch process, or may comprise a flow channel. One such apparatus comprises:
(A) a fluid flow conduit having a first region having a first cross-sectional area and an adjacent second region having a cross-sectional area that is less than said first cross-sectional area;
(B) a biocompatible pump in fluid communication with said fluid flow conduit, operable to pump fluid from said first region to said second region; and
(C) a photodetector in optical communication with said second region of said fluid flow conduit.

The fluid flow conduit can be any suitable shape, such as a tube, having a substantially circular or ellipsoidal cross section. In various embodiments, the fluid flow conduit, made from or lined with a biocompatible material. The material can be any useful material known biocompatible in the art. For example, any of the polyolefins, fluoropolymers, polyesters, polyamides, polyhydroxyalkanoates, polysulfones, glasses, and ceramics that are biocompatible can be used. In some embodiments, the biocompatible material can be coated, on the internal surface of the channel, with cell-attachment factors and/or other cell-supporting biomolecules; the internal surface of the channel can, in some embodiments, be attached to cells colonized thereon, e.g., endothelial cells. In some embodiments, the channel (walls) or the fluid-facing side thereof can comprise a hollow fiber format, e.g., a polysulfone that is capable of being attached by endothelial cells. One example of suitable material is the polysulfone hollow fiber PS+ material available from FiberCell Systems, Inc. (Frederick, Md., USA).

In various embodiments, at least one point along the fluid flow path defined by the conduit, can be located either (1) a stationery constriction of or deflection in the fluid flow conduit, or (2) a flexible wall of the fluid flow conduit to which pressure can be applied to form a constriction of or deflection in the fluid flow channel. A pump is operatively attached to the fluid flow conduit to permit circulation of an erythrocyte suspension therein. As cells of the suspension are physically distorted at the constriction or deflection, the erythrocytes can release ATP. In various embodiments, a photodetector flow cell can be present at or about the location of the construction or deflection. In various embodiments, the photodetector (and recorder) can be capable of detecting, and recording the amount of, light of about 560 nm when generated within the fluid flow channel. A luminometer can be used as the photodetector.

In various embodiments, the constriction or deflection can have an internal dimension (diameter) of about 1 to about 20 μm, or from about 1 to about 10 μm; the internal diameter can be at least 1, 2, 3, 4, or 5 μm, and/or up to 15, 10, 9, 8, 7, 6, or 5 μm. In some embodiments, the constriction or deflection can have an internal diameter of about 5 μm, or from 1 to about 5 μm. In some embodiments, the internal dimension thereof can be from about 10 to about 20 μm. The remainder of the flow conduit, i.e. the portion(s) that are not so constricted to deflected, can have an internal diameter of about or at least 50 μm; or up to or about 100 μm; or up to or about 200 μm.

In some alternative embodiments, no constriction or deflection need be provided, wherein that the flow channel has an internal dimension of about 100 μm or less, e.g., 50-100 μm. It is believed that the degree of shear stress experienced by RBCs passing through a passage of such a narrow diameter can be sufficient to initiate ATP release, even without a separate or distinct plasma membrane deformation-causing deflection or constriction. In other embodiments, mechanical energy may be provided to the RBCs to effect sufficient plasma membrane stress that ATP release occurs, e.g., possibly by squeezing a flexible vessel containing a luminescable, pretreated RBC sample or by shaking or otherwise disturbing a vessel containing such a sample.

The flow channel apparatus can further comprise a reservoir containing a supply of a cell-compatible, luciferin-containing solution. In various embodiments, the solution can have a pH of about 6.5 to about 8; in some embodiments, it can have a pH of about 7.8. In embodiments in which the detection enzyme is free-floating, the solution can further comprise luciferase, or luciferase can be added thereto at or about the time that an erythrocyte sample is combined therewith for loading into the assay apparatus for testing. The apparatus can further comprise a temperature control, e.g., a rheostat, to maintain the cell suspension at a cell-compatible temperature; in the case of human erythrocytes, this can be from about 36° C. to about 38° C., or can be about 37° C.

In a preferred embodiment, an apparatus for determining the level of erythrocyte ATP release of a test sample comprising erythrocytes, said apparatus comprises:
(A) a reservoir containing a supply of a cell-compatible, luciferin-containing solution;
(B) a biocompatible fluid flow conduit of approximately elliptical cross-section geometry and having, at a point along the fluid flow path, either (1) a stationery constriction of or deflection in the fluid flow conduit, or (2) a flexible wall of the fluid flow conduit to which pressure can be applied to form a constriction of or deflection in the fluid flow conduit; and
(C) a pump operative to distribute fluid along the fluid flow conduit; and
(D) a photodetector that is capable of detecting, and recording the amount of, light of about 560 nm when generated within the fluid flow conduit at or about said point(s) along the fluid flow path (B1 or B2);

whereby, upon introduction of said sample of erythrocytes and luciferase into the fluid flow conduit, operation of the apparatus can result in (1) generation of light of about 560 nm within the fluid flow conduit at or about said point(s) and (2) detection of light so generated, the detected amount of light thereby indicating the level of erythrocyte ATP release.

### Diagnostic and Therapeutic Methods

The methods and apparatus of the present technology can be used to perform a variety of different assays, based on the signal obtained from enzymatic reaction utilizing erythrocyte-released ATP, e.g., bioluminescence from luciferase. Thus, the present technology provides various therapeutic and diagnostic methods.

For example, an assay can be performed to determine the health status of a human or other animal subject, comprising assaying erythrocytes of the subject for their level of ATP release, and comparing that level to a normal range of ATP release determined under identical conditions for healthy individuals. Such an assay can be performed, so that, when a significantly reduced level of ATP release is found, as compared to the normal range, this can be used as indicator that that the subject likely has a disorder associated with abnormal levels of erythrocyte ATP release. Such disorders include sickle cell anemia, malaria, thalassemia, anemia, glucose processing disorders such as a diabetes or metabolic syndrome; chronic fatigue syndrome; or an obesity-related condition.

In some embodiments hereof, an apparatus can be used to assess the level of erythrocyte response modulation activity of an erythrocyte ATP-release response modulator. In some embodiments, an apparatus can be used to assess the efficacy of a treatment for an erythrocyte-membrane-altering pathological condition in a subject, comprising assaying erythrocytes of the treated subject for their level of ATP release upon physical deformation, and comparing that level: (A) to a normal range of ATP release, determined under identical conditions for healthy individuals; or (B) to an abnormal level of ATP release found in the pathological condition, determined under identical conditions for the untreated subject or for untreated others exhibiting the pathological condition; or (C) to both. If a significant change in the subject's ATP release level, to or toward the normal range (A) is found, this indicates that the treatment has a significant efficacy.

The present technology provides methods for modulating erythrocyte ATP-release response, methods for modulating glucose metabolism, and methods for promoting vasodilation in human or other animal subjects, comprising assaying erythrocytes of the subject for their level of ATP release. Such methods comprise, in various embodiments, administration of a safe and effective amount of erythrocyte ATP-release response modulators. Such methods and compositions useful herein are disclosed in PCT Pub. No. WO 2008/118387, Spence et al., published Oct. 2, 2008, incorporated by reference herein.

Although in many embodiments, ATP release modulators and assays therefore are providing or detecting increases in ATP release, in some embodiments hereof, conditions or substances that decrease RBC ATP release can be detected. The description of some embodiments hereof in the context of ATP release increase is not to be taken as a limitation on the usefulness of the present technology to monitor, and/or to detect compounds capable of causing, decrease in ATP release. Thus, in some embodiments, an assay hereof can be used to identify substances that are capable of decreasing RBC ATP release. Such assays can be used in some cases, e.g., to identify undesirable side effects of potential drug candidates.

Erythrocyte ATP-release modulators among those useful herein are compounds or complexes that are operable to increase the ability of the erythrocytes to release ATP. Without limiting the mechanism, function or utility of the present technology, in various embodiments, contacting erythrocytes with an erythrocyte ATP-release response modulator increases glucose uptake by the erythrocyte, with a concomitant increase in glycolysis and adenocyclase activity, thereby generating ATP. As a result, in some embodiments hereof, an erythrocyte response modulator can be employed to increase serum glucose clearance.

A “safe and effective” amount of an erythrocyte ATP-release response modulator is an amount that is sufficient to have the desired therapeutic effect in the human or other animal subject, without undue adverse side effects (such as toxicity, irritation, or allergic response), commensurate with a reasonable benefit/risk ratio when used in the manner of this technology. The specific safe and effective amount of the erythrocyte ATP-release response modulator will vary with such factors as the particular condition being treated, the physical condition of the patient, the nature of concurrent therapy (if any), the specific erythrocyte ATP-release response modulator used, the specific route of administration and dosage form, the carrier employed, and the desired dosage regimen.

In various embodiments, erythrocyte ATP-release response modulators are selected from the group consisting of pentoxifylline (1-(5-oxohexyl)theobromine), lisofylline (1-(5-hydroxyhexyl)theobromine), epoxidated arachidonic acids (e.g., 5,6-epoxy-eicosatrienoic acid), and salts and esters thereof; C-peptides and fragments thereof; mixtures of C-peptide or a fragment thereof and a source of a pharmaceutically acceptable polyvalent metal cation; complexes comprising a C-peptide or a fragment thereof and a polyvalent metal cation; and mixtures thereof. In some embodiments, two different types of erythrocyte response modulators can be administered to a subject, e.g., both such a compound and a C-peptide, fragment, or C-peptide complex. Specific compounds and compositions to be used in this technology must be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

C-peptide/polyvalent metal cation complexes useful herein comprise a C-peptide and a polyvalent metal cation, preferably a divalent or trivalent metal cation. Such a cation can also be co-administered with C-peptide to a subject, with complex formation taking place in vivo. In other embodiments, a C-peptide alone can be administered to a subject having an in vivo polyvalent metal cation composition that is sufficient for formation of a C-peptide complex in vivo.

As used herein, the term “C-peptide” refers to a polypeptide comprising an amino acid sequence of a C-peptide, preferably a native C-peptide, such as is produced during normal proinsulin processing to form insulin. Preferably, the sequence does not comprise an insulin A-chain or B-chain amino acid sequence, although in some embodiments, about 5 or fewer than 5 residues of one or both of these can be present. Native C-peptides typically are from about 26 to about 32 amino acid residues long. A “native” C-peptide refers to a C-peptide of a proinsulin molecule found in nature. SEQ ID NOs:2-7, 9, and 11-37 present examples of useful native C-peptide amino acid sequences. In various embodiments, the C-peptide of a C-peptide/Cr(III) complex hereof can have an amino acid sequence that is obtained from a species homologous to that of the subject to whom the complex is to be administered. A “homologous” amino acid sequence of a C-peptide hereof refers to an amino acid sequence that is at least 80% similar to that of a native C-peptide and that retains the acidic (i.e., Asp and/or Glu) residues of that native C-peptide. In some embodiments, such a homologous amino acid sequence can be at least 80% identical to the native sequence, i.e. while retaining the acidic residues thereof. In various embodiments, the homologous amino acid sequence can be at least or about 85, 90, or 95% similar or identical to the native sequence; in some embodiments, the homologous amino acid sequence can be at least 81, 84, 87, 93, or 96% similar or identical to the native sequence.

The composition and methods of the present technology may comprise a C-peptide fragment. In general, references to “C-peptide” herein are to include C-peptide fragments, which may be used in the compositions and methods of this technology in combination with, or instead of, a C-peptide. As referred to herein, a “fragment” is a peptide comprising amino acid residues that consist of a portion, but not the entirety, of a C-peptide or a homolog thereof, as described above. Thus, in various embodiments, a fragment may comprise less than about 26 to 32 amino acid residues. Fragments may comprise 20 or less, 15 or less, or 10 or less residues. Fragments may comprise 5 or more, 10 or more or 15 or more residues. Examples of fragments include SEQ ID NOs:38-45, set forth in the table, below. Fragments may comprise substitutes of amino acids found in C-peptides. The order of amino acids within fragments may also be altered from those in a C-peptide, such as SEQ ID NO:45. In various embodiments, a fragment comprises a peptide comprising the residue of SEQ ID NO:38.

C-PEPTIDE FRAGMENTS

| SEQ ID NO | TITLE | SEQUENCE |
|---|---|---|
| 38 | C-peptide | GLU-GLY-SER-LEU-GLN |
| | residues 27-31 | |
| 39 | C-peptide | SER-LEU-GLN-PRO-LEU-ALA- |
| | residues 20-31 | LEU-GLU-GLY-SER-LEU-GLN |
| 40 | C-peptide | VAL-GLU-LEU-GLY-GLY-GLY- |
| | residues 10-31 | PRO-GLY-ALA-GLY-SER-LEU- |
| | | GLN-PRO-LEU-ALA-LEU-GLU- |
| | | GLY-SER-LEU-GLN |
| 41 | C-peptide | GLU-LEU-GLY-GLY-GLY-PRO- |
| | residues 11-19 | GLY-ALA-GLY |
| 42 | C-peptide | GLU-ALA-GLU-ASP-LEU-GLN- |
| | residues 1-13 | VAL-GLY-GLN-VAL-GLU-LEU- |
| | | GLY |
| 43 | C-peptide | ALA-GLY-SER-LEU-GLN |
| | residues 27-31 | |
| | (E27A) | |
| 44 | C-peptide | ASP-GLY-SER-LEU-GLN |
| | residues 27-31 | |
| | (E27D) | |
| 45 | C-peptide | SER-GLN-LEU-GLU-GLY |
| | residues 27-31 | |
| | (scrambled) | |

In various embodiments, the C-peptide is combined in vitro or in vivo with a pharmaceutically acceptable polyvalent metal cation; in some embodiments, this can be a divalent or trivalent metal cation. Such cations include: divalent Mg, Ca, Sr, Ba, Ge, or Sn cations; trivalent Al, Ga, In, or Bi cations; di- or tri-valent transition metal cations; and di- or tri-valent lanthanide (La, Ce, Pr, Nd, Pm, Sm, Eu, Gd, Tb, Dy, Ho, Er, Tm, Yb, or Lu) cations; and combinations thereof. The cation can be a polyvalent transition metal cation or a combination thereof. In some embodiments, the cation can be a polyvalent Cr, Mn, Fe, Co, Ni, Cu, Zn, Mo, Ag, Pt, or Au cation, or a combination thereof. In some preferred embodiments, a polyvalent Cr, Mn, Fe or Zn cation, or a combination thereof, can be used; or Cr(III) and/or Fe(II); or Cr(III); or Zn (II).

In some embodiments, metal complexes can include a combination of polyvalent metal cations or one or more monovalent metal cations, e.g., alkali metal cations. Complexes can comprise, in addition to the metal cation(s) and C-peptide, one or more further pharmaceutically acceptable, mono- or di-valent anions, or electron donors. Such anions include halide, oxyacid, and other anions, including those commonly found in commercially available Cr(III) salts, such as esters, halides (e.g., chloride or bromide), and physiologically acceptable acids, including carboxylic acids (e.g., polycarboxylic acids), amino acids, sulfoxy acids (e.g., sulfate, bisulfate, sulfonate), phosphoxy acids (e.g., phosphate, biphosphate, phosphonate, biphosphonate), carbonate, bicarbonate, nitrate, aromatic acids, nucleoside phosphates, and their esters. In various embodiments, the c-peptide/polyvalent metal cation complexes comprises from about 10 to about 67 mole percent polyvalent metal cation, based on the total moles of ions present in the complex.

Examples of chromium complexes and salts include: chromium picolinate, chromium citrate, chromium chloride, chromium aspartate, Cr-ATP complexes (e.g., Cr-ATP-Cys₂), Cr-ADP complexes, chromium trinicotinate, chromium dinicotinate chloride, Glucose Tolerance Factor (GTF), and the like. At physiological pH, GTF is reported to comprise Cr(III) complexed with one O-glutathionyl ligand and two O-nicotinyl ligands. Such electron pair donors and anions are also useful in forming mixed complexes containing Cr(III) and C-peptide. In some embodiments, the anions or electron donor(s) present in such metal compounds can be selected for use as a further component in a C-peptide complex hereof.

The C-peptide/polyvalent metal cation complex or other erythrocyte ATP-release response modulator may be used in a composition additionally comprising a pharmaceutically-acceptable carrier. Such compositions can be in any suitable dosage form, such as for enteral, parenteral, or topical administration. The specific carrier may comprise one or more materials, and may be adapted for the intended route of administration for the composition. Such carrier materials may include, for example, diluents, lubricants, binders, solvents, dissolution promoters, buffers, preservatives, flavorants, sweeteners, and colorants. In particular, for example, transdermal formulations can comprise skin-enhancing agent(s), enteral formulations for oral administration can comprise a flavoring, viscosity modifier, or mouth-feel-improving agent, and formulations for nasal administration can comprise a scent.

In some embodiments, an ATP-release response modulator can be further combined with other bioactive agents. Such bioactive agents can be, for example, pharmaceutical, nutraceutical, or nutritive agent(s). In some embodiments, a further pharmaceutical agent can be included and this can be a small molecular or biomolecular pharmaceutical.

In various embodiments, the compositions comprise a glucose metabolism modulator. Glucose metabolism modulators useful herein include insulin, hypglycemic agents, and mixtures thereof. As referred to herein, “insulin” includes native insulin as well as naturally-occurring and synthetic analogs of insulin as are known in the art. Hypoglycemic agents include oral agents such as tolbutaminde, chlorpropamide, tolazamide, acetohexamide, glyburide, glipizide, gliclazide, and mixtures thereof.

In various embodiments, the present technology provides methods for promoting glucose clearance or vasodilation in a human or animal subject, comprising administering to the subject a safe and effective amount of a pharmaceutically acceptable C-peptide/metal cation complex in which the metal cation comprises a pharmaceutically acceptable M(II) or M(III) cation or other erythrocyte ATP-release response modulator. Such methods for promoting glucose metabolism may be performed in subjects having diabetes mellitus type 1, diabetes mellitus type 2, gestational diabetes, or metabolic syndrome. The method may be a prophylactic treatment for a subject identified as being at risk for developing a disorder of glucose processing, or a palliative treatment for a subject having a glucose processing disorder.

The present technology also provides regimens for treating diabetes mellitus in a human or other animal subject comprising administering to the subject a glucose metabolism modulator and erythrocyte ATP-release response modulator, wherein said erythrocyte ATP-release response modulator is effective to reduce the level of the glucose metabolism modulator needed to effect glucose control in the subject, extend the duration of efficacy of the glucose metabolism modulator in the subject, or both. The glucose metabolism modulator may be, for example, insulin or a hypoglycemic agent. In various embodiments, the erythrocyte ATP-release response modulator and glucose metabolism modulator are administered at “synergistic” levels. In such methods, the therapeutic effect of administering of the combination of the erythrocyte ATP-release response modulator and glucose metabolism modulator is greater than the additive effect of administering erythrocyte ATP-release response modulator and glucose metabolism modulator individually. Such effects include one or more of increasing the effect of the glucose metabolism modulator, increasing the duration of the effect of the glucose metabolism modulator, and making glucose metabolism modulator effective at dosage levels that would otherwise be ineffective.

In various embodiments hereof, erythrocyte ATP-release response modulators are provided that can be administered to a subject in order to increase vasodilation or the vasodilation potential of RBCs, and/or to increase glucose clearance from serum by enhancing glucose uptake by RBCs. In some embodiments, an erythrocyte response modulator can be used to treat a vascular condition, such as, but not limited to: hypertension; gestational hypertension; peripheral vascular diseases; chronic venous insufficiency; Raynaud's disease; such conditions in other disorders, e.g., Raynaud's involvement in scleroderma, lupus, Sjögren's syndrome, or rheumatoid arthritis; and vascular aspects of cardiac care, of recovery following heart failure, of stroke, of recovery following stroke, or of erectile dysfunction. In some embodiments hereof, an erythrocyte response modulator can be used to treat a glucose processing disorder, such as, but not limited to: diabetes mellitus type 1 or type 2, gestational diabetes, hyperglycemia, or metabolic syndrome. An erythrocyte modulator may also be used to treat other disorders, such as those associated with RBC membrane described above, e.g., malaria, chronic fatigue syndrome, and obesity.

### Methods of Screening Pharmaceutical Actives

The present technology also provides methods for screening substances to identify a candidate erythrocyte ATP-release response modulator(s). Thus, methods are provided for screening substances to identify a candidate erythrocyte ATP-release modulator, comprising
(A) providing a test substance and a sample of erythrocytes
(B) contacting a first portion of said sample of erythrocytes with said substance to prepare treated erythrocytes;
(C) assaying said treated erythrocytes for their level of ATP release to obtain a treated erythrocyte ATP release assay level;
(D) assaying a second portion of said sample of erythrocytes to obtain a control erythrocyte ATP release assay level; and
(E) comparing said treated erythrocyte ATP release assay level with said control erythrocyte ATP release assay level.

In various methods the sample of erythrocytes comprises erythrocytes obtained from a plurality of samples of erythrocytes having characterized ATP release characteristics, such that statistically meaningful comparison of said treated erythrocyte assay level and said control erythrocyte ATP release assay level may be made without concomitantly performing the steps of assaying said treated erythrocytes and assaying said second portion. In some methods, the control ATP release assay level is a reference standard level determined by repeating the assaying of the second portion on a plurality of second portions.

A library of compounds can be tested utilizing such an assay. Each compound is contacted with erythrocytes prior to the assay. A compound can be contacted with the cells for a few minutes, up to a few hours or, e.g., 1 to 2 days. The treated erythrocytes are then assayed for ATP release and this is compared to a level of ATP release determined under identical conditions for untreated erythrocytes. Determination that a given test compound has significantly increased the level of ATP release by erythrocytes, thus identifies the test substance as a candidate erythrocyte ATP-release response modulator. Further tests can be employed separately to determine if the identified modulator is pharmaceutically acceptable.

### EXAMPLES

### Materials & Methods

Genetic techniques can be performed according to commonly known methods of nucleic acid manipulation, such as those described in: F M. Ausubel et al. (eds.), Current Protocols in Molecular Biology (2006) (Wiley Interscience, NY); F. M. Ausubel et al., Short Protocols in Molecular Biology (2002) (5^{th} ed.; John Wiley & Sons); Sambrook & Russell, Molecular Cloning: A Laboratory Manual (2001) (Cold Spring Harbor Lab., NY); W Ream & K Field, Molecular Biology Techniques: An Intensive Laboratory Course (1998) (Academic Press); C. R. Newton & A. Graham, “PCR,” in series Introduction to Biotechniques (1997) (2^{nd} ed.; Springer Verlag); and Berger & Kimmel (eds.), “Guide to Molecular Cloning Techniques,” in series Methods in Enzymology 152 (1987) (Academic Press, San Diego, Calif.).

Pharmaceutical formulations for administration can be prepared by any useful method known in the art, such as those described in: A. R. Gennaro et al., Remington: The Science and Practice of Pharmacy (2005) (21st ed.; Lippincott Williams & Wilkins, Phil., Pa.) (Univ. Sci. in Phil., Pa.); R. C. Rowe et al., Handbook of Pharmaceutical Excipients (2005) (APHA Publications, Washington, D.C.); L. Brunton et al., Goodman & Gilman's The Pharmacological Basis of Therapeutics (2005) (11^{th} ed.; McGraw-Hill Professional, New York, N.Y.); and S. K. Niazi, Handbook of Pharmaceutical Manufacturing Formulations (2004) (Informa Healthcare, London, UK) (esp. vol. 2).

Preparation of RBCs. Rabbits (New Zealand whites, males, 2.0-2.5 kg) were anesthetized with ketamine (8 ml/kg, i.m.) and xylazine (1 mg/kg, i.m.) followed by pentobarbital sodium (15 mg/kg i.v.). A cannula was placed in the trachea and the animals were ventilated with room air. A catheter was placed into a carotid artery for administration of heparin and for phlebotomy. After heparin (500 units, i.v.), the animals were exsanguinated. Human blood was obtained by venipuncture without the use of a tourniquet (antecubital fossa) and collected into a heparinized syringe. Blood was centrifuged at 500×g at 4° C. for 10 min. The plasma and buffy coat were discarded. The RBCs were resuspended and washed three times in a physiological salt solution [PSS, containing in mM: 4.7 KCl, 2.0 CaCl₂, 140.5 NaCl 12 MgSO₄, 21.0 tris(hydroxymethyl)aminomethane, 11.1 dextrose with 5% bovine serum albumin (final pH 7.4)]. Cells were prepared on the day of use and experiments were finished within 8 hours of removal from the animal or human subjects. All procedures were approved by the Animal Investigation Committee or the Human Investigation Committee at Wayne State University.

Measurement of ATP. Human C-peptide (American Peptide Co., Sunnyvale, Calif.), 0.25 mg (MW=3020 g/mol), was dissolved in 100 mL of purified water (18.2 megaohm) to yield a concentration of 83 μM. Next, an appropriate volume of this C-peptide solution was added to 10 mL of a 7% solution of RBCs to create a solution containing the C-peptide at concentrations in the 1-5 nM range. The RBC-peptide solution was immediately loaded into a 500 μL syringe and placed on a syringe pump; the other syringe contained a solution of luciferin/luciferase (Sigma, FLE-50 with 2 mg of added luciferin to improve sensitivity). Both solutions were pumped simultaneously at a rate of 6.70 μL/min through portions of fused-silica microbore tubing (50 μm i.d., 365 μm o.d., Polymicro Technologies, Phoenix, Ariz.) to a mixing tee. The resulting chemluminescence reaction flowed through a final potion of fused-silica microbore tubing that was placed over a photomultiplier tube, where the emission was detected. The resultant current was measured as a potential by a data acquisition board operated by a program written with the LabView software package (National Instruments, Austin, Tex.).

To ensure that the resulting increase in ATP release was due to C-peptide interacting with the RBCs and not cell lyses, the RBC solutions were measured under non-flow conditions using a luminometer with 200 μL of the RBC solution and 200 μL of the luciferin/luciferase solution. No detectable signals were obtained. To ensure that lysis was not occurring in the tubing, a solution of 0.01 M glybenclamide was prepared by adding 49 mg of glybenclamide (Sigma) to 2 mL of a 0.1 M solution of sodium hydroxide. To this, 7.94 mL of a dextrose solution (1 g dextrose in 20 mL of purified water) was added. The mixture was heated carefully to 52° C. until all of the glybenclamide was dissolved. Once the solute was completely dissolved, 1 mL of this solution was added to 9 mL of PSS, resulting in a solution with a concentration 0.001 M. From this diluted solution, 2.5 mL were added to 2.5 mL of 7% hematocrit RBC solution, resulting in a 3.5% hematocrit solution of RBCs. This solution was allowed to incubate for 15 minutes. As a comparison, 2.5 mL of wash buffer without glybenclamide was added to 2.5 mL of 7% hematocrit RBCs. After 15 minutes, the RBC solutions were assayed.

Introduction. C-peptide may be able to mediate the production of endothelium-derived NO via its ability to increase the levels of ATP released from erythrocytes that are subjected to mechanical deformation. Here, studies are performed in which RBCs are pumped through microbore tubing having diameters that approximate those of resistance vessels in vivo. Upon deformation in the tubing, the RBCs release ATP that is measured using a well-established chemiluminescence assay for ATP. The concentrations of RBC-derived ATP are measured in the presence and absence of synthetic C-peptide. Mass spectrometric data unexpectedly reveals that binding of the C-peptide to a polyvalent metal cation, here using chromium (III), is necessary for extended activity of the peptide.

### Example 1

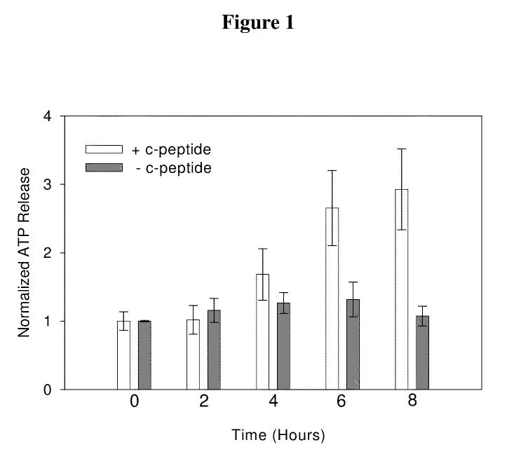

C-Peptide-Induced Release of ATP. RBCs obtained from rabbits are pumped through microbore tubing having an inside diameter of 50 μm and the resultant ATP released by the cells upon deformation in the tubing is measured. See, J. S. Carroll et al., in Mol. Biosys. 2:305-311 (2006); R. Sprung et al., in Anal. Chem. 74:2274-2278 (2002). Another aliquot from the same RBC sample is incubated in 1 nM C-peptide and the resultant ATP release measured every 2 h for a period up to 6 h. As shown in FIG. 1, which contains normalized values of ATP released from the RBCs of n=11 rabbits, the C-peptide has the ability to increase the deformation-induced release of ATP from the RBCs. The data shown are normalized values from the RBCs of n=12 rabbits incubated in the presence and absence of 1 nM c-peptide. As shown, the ATP release (determined by a chemiluminescence assay) from those cells incubated in the c-peptide increased approximately 2.9 times over a period of 8 h. RBCs in the absence of the c-peptide demonstrated to statistically significant change in their ability to release ATP. Error bars are ±SEM. The increase seen over the 6 h period is nearly three times that of the RBCs incubated with a control (buffer without C-peptide). In addition, the increase in the ATP release can be inhibited when the RBCs are incubated in glybenclamide, a substance known to inhibit ATP release from RBCs. This inhibition demonstrates that the increase in measured extracellular ATP is not due to cell lysis. If cell lysis were occurring, the glybenclamide would have no affect on the measured ATP as it would be present in extracellular form whether or not glybenclamide was introduced to the RBCs.

### Example 2

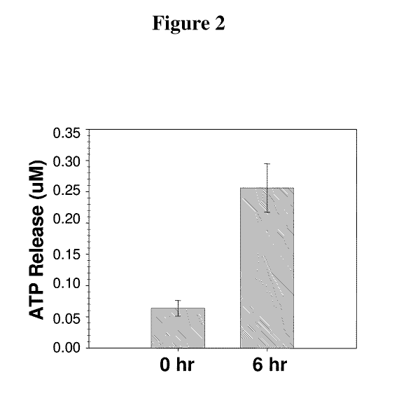

Restoration of ATP Release from the RBCs of Patients with Diabetes. Recently, it has been reported that RBCs obtained from the whole blood of patients with Type II diabetes mellitus release approximately 50% of the ATP released from the RBCs of healthy control patients. Thus, RBCs of diabetic patients may have released less ATP due to oxidative stress within the RBCs, leading to a less deformable cell. A decrease in RBC deformability is a recognized trait of the RBCs obtained from patients with diabetes. See, L. O. Simpson, in Nephron 39:344-51 (1985); R. S. Schwartz et al., in Diabetes 40:701-712 (1991). The ability of C-peptide to restore ATP release in diabetic RBCs is assayed. As shown in FIG. 2, C-peptide administration is now been found to have the ability to increase the ATP release from the RBCs of patients with type II diabetes (n=7). Moreover, this effect is substantial in that such administration has the ability to restore these release levels to a value that is statistically equivalent to that of healthy, non-diabetic control patients.

### Example 3

Mass Spectrometric Analysis of Metal-Peptide Binding. Additional results from repeats of Experiments 1 and 2 initially and unexpectedly failed to confirm the ATP-release modulating effect of C-peptide. Metaanalysis of the collective data surprisingly revealed that a C-peptide preparation would generally lose bioactivity about 24-36 h after preparation in water. Analysis of the C-peptide using electrospray ionization mass spectrometry indicates that the peptide is not undergoing any type of degradation or cleavage, even after remaining in solution for periods >30 days. Thus, alternative postulated causes are tested, including that a covalent modification of the peptide, e.g., induction or lysis of a side-chain-to-side-chain bond or of a moiety covalently attached to an amino acid residue, might be involved, or that non-covalent interaction with another chemical species, e.g., a metal ion, might be involved in this effect.

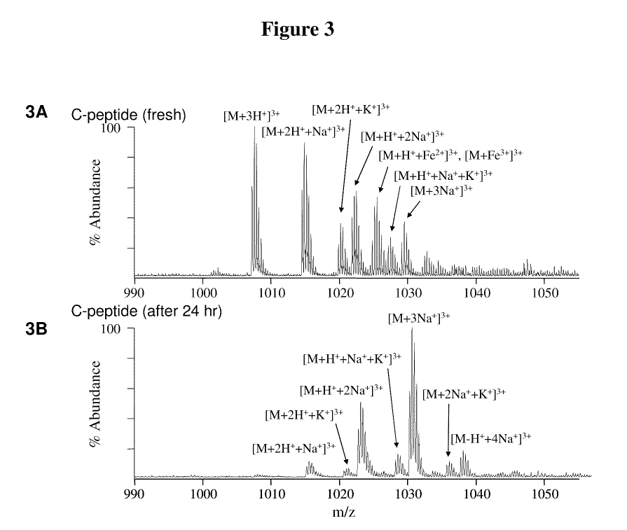

The data in FIG. 3 reveal some information about the possible loss of activity of the C-peptide after preparation in the aqueous solvent. Specifically, the mass spectrum shown in FIG. 3a is that of peptide prepared in water and analyzed within 0.5 h of preparation. In 3a, the [M+3H]³⁺ peak is present as are other forms of the peptide with sodium atoms, potassium atoms, or a combination thereof. Interestingly, there is also a peak that corresponds to binding to an iron atom [M+H⁺+Fe²⁺]³⁺. The presence of this Fe(II) adduct to the C-peptide is not present 24 h after preparation. These data provide evidence suggesting that the activity of the peptide involved binding to metal cation(s).

### Example 4

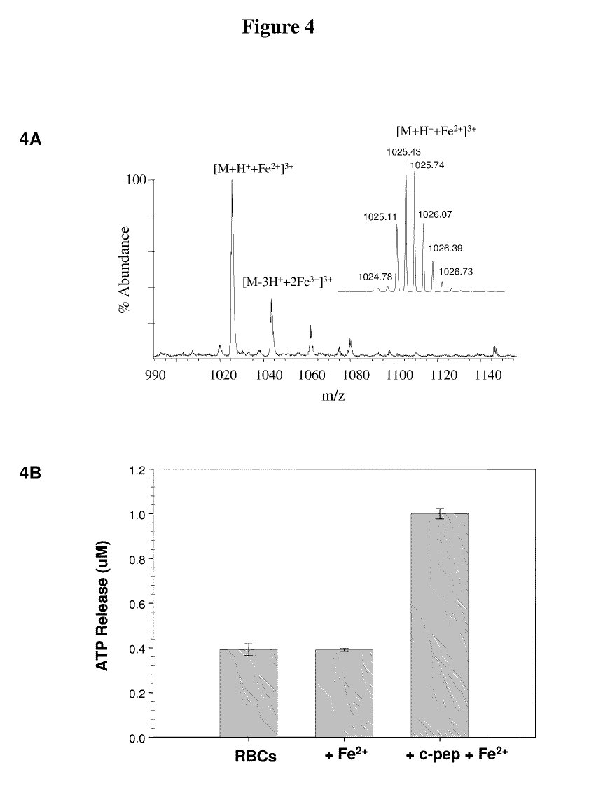

Metal-Induced Activity of C-Peptide. Based on the data shown in FIG. 3, which demonstrates the ability of the C-peptide to bind to Fe(II), RBCs are incubated in solutions containing Fe(II) and their subsequent ability to release ATP upon being subjected to deformation is determined. The data in FIG. 4 is consistent with the data shown in FIGS. 1 and 2; namely, that the activity of the C-peptide is dependent upon its ability to bind to the metal ion. Specifically, RBCs are incubated in C-peptide that has been kept at 4° C. for >30 days; therefore, this solution of C-peptide no longer has the ability to induce ATP release from deformed RBCs. This same inactive C-peptide solution is then combined with an Fe(II) source such that the concentrations of both Fe(II) and C-peptide are 1 nM. This solution containing C-peptide and Fe(II) is then applied to the RBCs and, after 6 h, the RBC-derived ATP is measured. The results in FIG. 4 clearly demonstrate that the activity of the C-peptide can be restored when bound to the Fe(II) metal ion. As a control, the RBCs are incubated with the metal ion in the absence of the peptide and it is found that the solution of metal ion alone does not result in an increase in RBC-derived ATP.

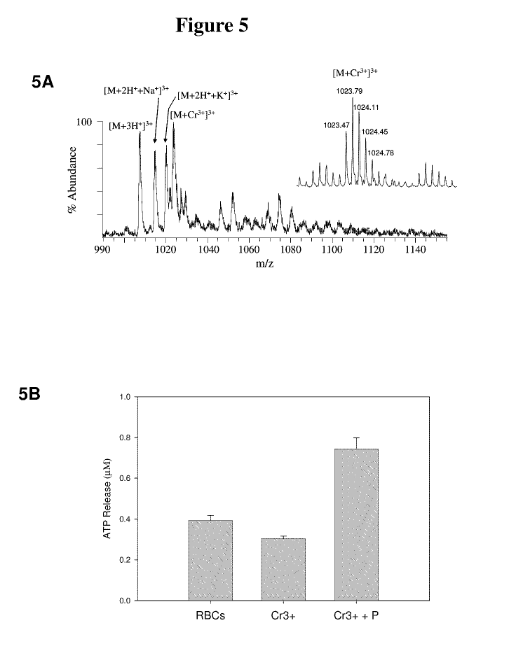

Although the Fe(II)-bound C-peptide has the ability to increase ATP-release from deformed RBCs, its activity also appears somewhat limited. Specifically, while the addition of Fe(II) to inactive C-peptide is able to restore the peptide's activity, it too decreases after 24 h. Moreover, it is found that, beyond 48 h, the activity of the Fe(II)-bound C-peptide generally shows no statistical difference from that of C-peptide alone. Mass spectrometric examination of the Fe(II)-C-peptide adduct, shown in FIG. 5, was found to help explain this observation. The unexpected result is that the population of Fe(II)-C-peptide adduct begins to diminish within 24 h after the addition of an Fe(II) source, and Fe(II) is then replaced by either sodium or potassium, or both, cations in the C-peptide complex.

### Example 5

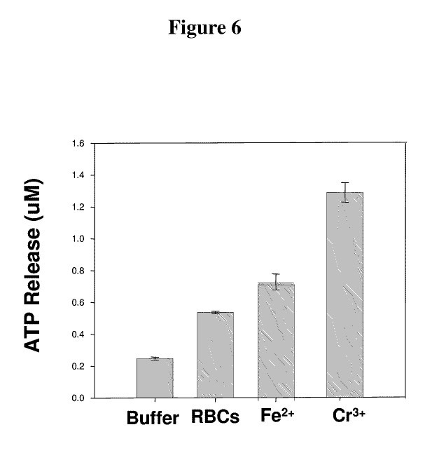

Improving Metal-Induced Activity of C-Peptide. In order to extend the activity of the C-peptide, other metal cations are tested. For example, a chromium (III) source is added to a solution of inactive C-peptide. The data in FIG. 6a show that the Cr(III) is able to bind the C-peptide. The measured mass spectrometric signal of this adduct is found to be more stable than the Fe(II)-C-peptide adduct (cf. FIG. 5). The C-peptide/Cr(III) adduct is also tested for erythrocyte ATP-release bioactivity. FIG. 5b shows that Cr(III) alone does not result in any significant increase in ATP release from deformed RBCs. However, when the C-peptide/Cr(III) adduct is added to a suspension of RBCs, ATP release occurs in a manner similar to that shown in FIG. 4 for the Fe(II)-C-peptide complex.

### Example 6

Extension of C-peptide Activity through Binding to Cr(III). To determine the longevity of bioactive C-peptide/metal ion complexes, aliquots from an inactive solution of the C-peptide are mixed with equimolar amounts of either Fe(II) or Cr(III) and allowed to incubate for 48 h. After this incubation period, the C-peptide/metal cation mixtures are introduced into a fresh RBC suspension and allowed to incubate in the RBCs for 6 h. These cells are then mechanically deformed in order to measure the RBC ATP-release response. The data in FIG. 6a reveals that the activity of the C-peptide bound to Cr(III) results in a significantly greater ATP-release response from the RBCs. Furthermore, the results in FIG. 6b also indicate that the activity of the C-peptide in complex with Cr(III) is extended to periods beyond 4 days. These results for C-peptide/metal cation complex longevity are based on residence times in aqueous solution. However, frozen or lyophilized preparations would generally provide much greater complex longevity, as would preparations, e.g., concentrates, containing an excess of the Cr(III), Fe(II), or other desired polyvalent cation in the presence of little or no monovalent cation content.

### Example 7

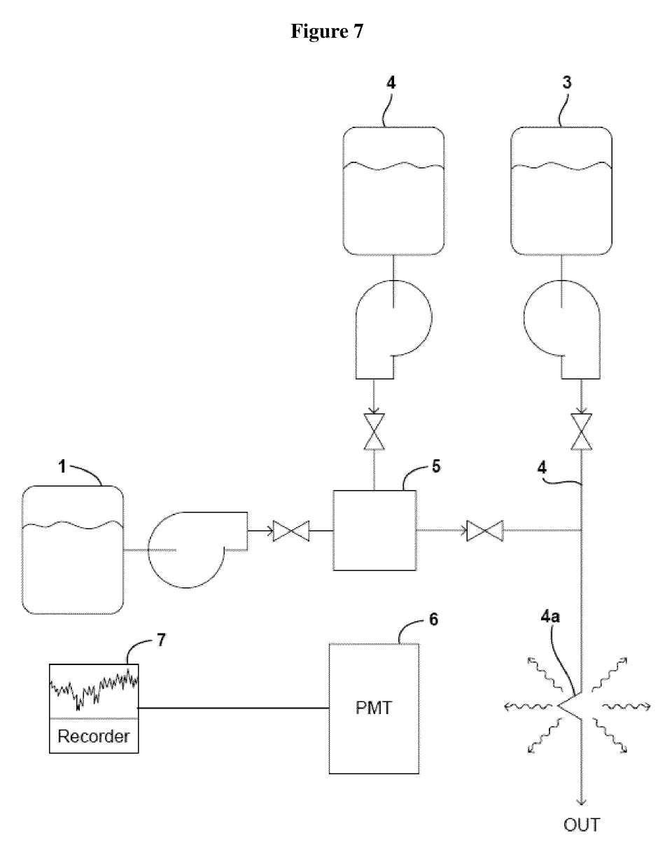

Formats for Apparatus Useful in RBC ATP Release Assays. FIGS. 7, 8, and 9 illustrate some embodiments of a device that can be used herein. In FIG. 7, a schematic is shown in which RBC suspension (1) is tested, though a network (4) of channels/tubing, pumps, and valves. Modulator or test compound from solution (2) is pumped into pre-treatment chamber (5) with RBCs from suspension (1). After a desired time, resulting treated RBCs are delivered through the network (4) to a stream of luciferin/luciferase solution (3) wherein the RBCs can exhibit luminescence at a predetermined locus (4a). In some embodiments, this locus comprises a deflection or constriction that is operative to cause physical deformation of RBC plasma membranes. Light emitted at or about locus (4a) is detected by detector (6), e.g., a PMT, and the detected signal is transmitted to device (7) for recording.

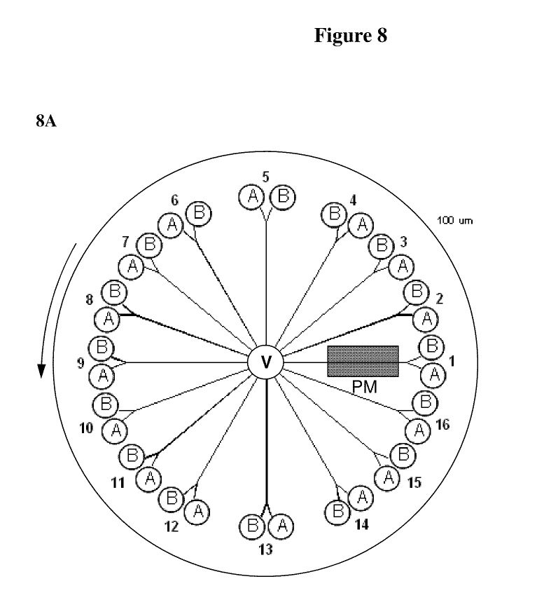

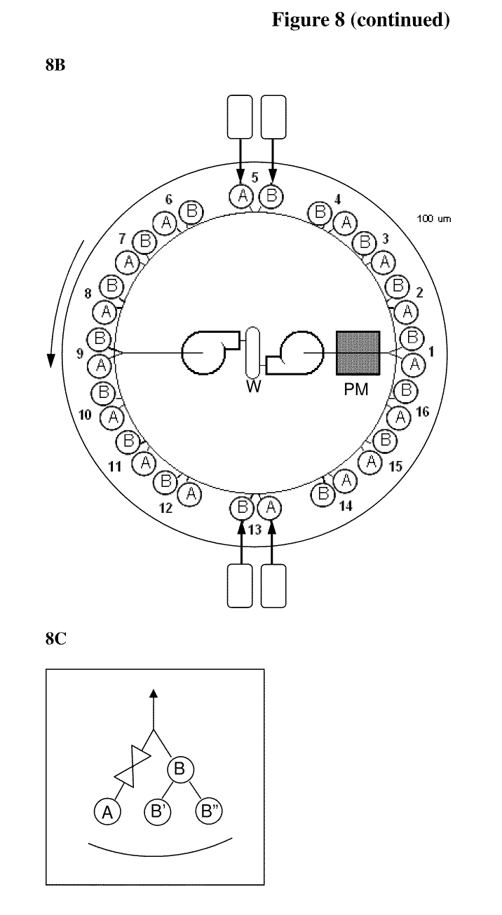

FIG. 8 illustrates embodiments of a rotating “chip” designed to allow high-throughput of samples in which a flow channel deviation or constriction is optional. As shown, the Y-shaped channels of these exemplary devices are 100 μm in internal diameter. In some embodiments, sample wells and channels can be punched or carved into, or molded within, polydimethylsiloxane layers. Chemiluminescence measurements are taken from each channel that is placed over a PMT.

In a rotating plate device, FIG. 8A, each of the wells, paired A and B, can be loaded, e.g., by operation of a vacuum aspirator, with, e.g., modulator-treated RBC samples being loaded into wells B, and a luciferin/luciferase solution being loaded into wells A. When a pair of wells has rotated into position such that their common channel is located over the detection device (PMT), an aspirator draws the contents of the wells together into their common channel for detection. A plate can be disposable or re-usable.

In a rotating ring device, FIG. 8B, samples can similarly be loaded into rotating well pairs, from positions shown at 13A and 13B; rotation to the position shown at 1A and 1B brings the ports of each channel into operative alignment with the stationary channel over the PMT, whereupon a pump applies vacuum aspiration to draw in and mix the fluids for detection, ultimately sending them to waste (W). In some embodiments, the wells can be reused, as by rotation to the position shown at 5A and 5B at which point the wells can receive a washing solution that is later removed by vacuum aspiration at the position shown at 9A and 9B. In some embodiments, the ring can be removable and replaceable with other ring(s).

In some alternative embodiments, such as that illustrated in FIG. 8C, four wells can be arranged together, rather than two. In such an embodiment, Wells B′ and B″ can be loaded with RBCs and an ATP release modulator/test compound, respectively. These can be pre-combined in some embodiments in well B by vacuum aspiration, with either a separate valve provided to isolate well A during aspiration of B′ and B″ into well B, or with a subsequent step utilized for loading well A. The RBCs are thereby pre-treated in well B. Subsequent operation is then as described above.

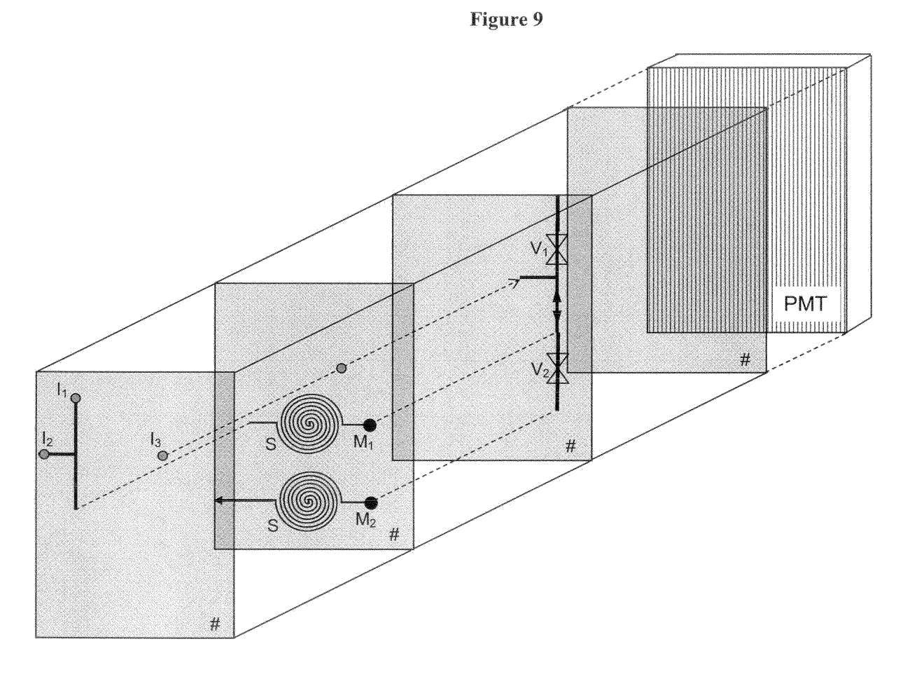

FIG. 9 illustrates some embodiments of a chip design in which multiple layers of a substrate, e.g., polydimethylsiloxane (PDMS), can be formed to provide ports and channels useful to test RBC ATP release. Depicted are: Plate 1 having a T-shaped channel located as shown on its reverse side and input ports I1, I2, and I3 punched therethrough; Plate 2 having one or two spiraling channels located therein or therethrough, with spiraling channel S1 being operatively attached to a 3-5 mm diameter hole, M1, punched through Plate 2, with M1 serving as a mixing chamber for combining a luciferase solution with a treated-RBC sample; Plate 3 having a T-shaped channel located as shown therein or therethrough, for receipt of a luciferin/luciferase solution, and valved port V1 to permit venting, and optionally having an extension of the channel, through optional valve V2, that is in operative alignment with the optional mixing chamber M2 and optional spiraling channel S2 located in Plate 2; and Plate 4 as a backing layer. The resulting chip can be placed over a detector shown as PMT.

In some embodiments, Plates 1 and 3 can be constructed of, e.g., 5:1 (softer) polydimethylsiloxane, with Plates 2 and 4 being of 20:1 (harder) polydimethylsiloxane. In such an embodiment, the chip can be prepared as follows. Mold all channels, including S1 (and optionally S2) during formation of layers. Then, separately, bake Plates 1 and 3 at 75° C. for 25 min, and Plates 2 and 4 for 30 min at that temperature. Then punch inlet holes I1, and I2 using a 20 gauge Luer stub, and mixing chamber M1 (and optionally M2) using a ⅛-inch holepunch, punching from reverse face to obverse face. Remove cut-outs. Then place Plate 1 on Plate 2 and bake together for 20 min. Then place the two-layer assemblage over Plate 3, with the ⅛″ hole(s) in alignment with the channel of Plate 3 as shown, and bake all three layers together for 20 min. Then, using a 20 gauge Luer stub, punch inlet I3 from reverse to obverse direction through all three layers and remove the cut-out. Then place the three-layer assemblage onto Plate 4 bake together for 1 hour.

In operation, valved hypodermic tubing can be used to deliver an RBC sample to I1, a release modulator/test compound to I2, and a luciferin/luciferase solution to I3. Positive pressure or vacuum can be used to draw the I1 and I2 solutions together though S1 and into and/or just past M1, at which point the direction of flow is reverse to permit the I3 solution to mix with the now pre-treated RBCs for luminescence during transit back through S1. Alternatively, the RBCs were pretreated during forward transit through S1 and drawn through V2, with the I3 solution, to M2 and then through S2 for bioluminescence in transit therethrough.

The embodiments and the examples described herein are exemplary and not intended to be limiting in describing the full scope of compositions and methods of the present technology. Equivalent changes, modifications and variations of some embodiments, materials, compositions and methods can be made within the scope of the present technology, with substantially similar results.

What is claimed is:

1. A method for assessing the health status of a human or other animal subject, comprising performing an ATP release assay on erythrocytes of said subject to obtain an ATP release assay level, and comparing said assay level to a reference level of ATP release.

2. A method according to claim 1, wherein said reference level is a normal range of ATP release determined by assaying erythrocytes of normal subjects under conditions substantially identical to said assaying of erythrocytes of said subject.

3. A method according to claim 2, further comprising performing an additional diagnostic test for glucose processing disorder, chronic fatigue syndrome, or an obesity-related condition in said subject, if said assay level is significantly below said reference level.

4. A method according to claim 3, wherein said additional diagnostic test is for diabetes or metabolic syndrome.

5. A method according to claim 1, wherein said subject is at risk for developing diabetes.

6. A method according to claim 1, wherein said subject has been diagnosed with diabetes prior to said performing of the ATP release assay.

7. A method according to claim 6, wherein said subject is being treated for diabetes.

8. A method according to claim 7, wherein said subject has diabetes mellitus type 1.

9. A method according to claim 1, for diagnosing diabetes in said subject.

10. A method according to claim 1, for managing the health of said subject wherein said subject is at risk for diabetes.

11. A method according to claim 1, wherein said assaying comprises applying physical force to said erythrocytes.

12. A method according to claim 11, wherein said assaying comprises obtaining a suspension of said erythrocytes, applying said physical force to said suspension so as to deform said erythrocytes, and detecting ATP levels in said suspension.

13. A method according to claim 12, wherein said detecting comprises using luciferase.

14. A method according to claim 13, wherein said obtaining a suspension of said erythrocytes comprises admixing luciferin and a sample of said erythrocytes from said subject to form a suspension having a pH 6.5 to about pH 8, contacting said suspension with luciferase, and observing said suspension for the presence of luciferase-catalyzed luminescence.

15. A method according to claim 14, wherein said luciferase is immobilized on a surface that is in contact with said suspension.

16. A method according to claim 14, wherein said suspension has a pH of about 7.8.

17. A method according to claim 12, wherein the physical deformation is performed by applying pressure to a flexible wall of a vessel in which the suspension is located.

18. A method according to claim 17, wherein said applying of force comprises pumping said suspension through a conduit having a first region with a first cross sectional area and an adjacent second region having a cross sectional area less than said first cross sectional area, and said suspension is pumped from said first region into said second region.

19. A method according to claim 12, wherein said second region has an internal dimension of about 1 to about 20 microns.

20. A method according to claim 19, wherein the minimum internal dimension of said first region is of about or at least 50 μm.

21. A method according to claim 19, wherein said second region has an internal dimension of from about 1 to about 10 μm.

22. A method according to claim 1, wherein the method further comprises contacting the erythrocytes, prior to performing the ATP release assay, with an ATP-release modulator.

23. A method according to claim 22, wherein the ATP-release modulator is pentoxifylline, lisofylline, an epoxidated arachidonic acid; a salt or ester of any of these; a C-peptide or fragment thereof; a combination of a C-peptide, or a fragment thereof, and a polyvalent metal cation source; a complex comprising a C-peptide or a fragment thereof with a polyvalent metal cation; or a combination thereof.

24. A method according to claim 23, wherein the ATP-release modulator is a combination of a C-peptide and a polyvalent metal cation source, or a complex comprising a C-peptide or a fragment thereof and a polyvalent metal cation.

25. A method according to claim 24, wherein the polyvalent metal cation is Cr(III), Fe(II), Zn(II), or a combination thereof.

26. A method determining the efficacy of erythrocyte ATP-release activity of a compound, comprising
(A) contacting a sample of erythrocytes with said compound to prepare treated erythrocytes;
(B) assaying said treated erythrocytes for their level of ATP release to obtain a treated erythrocyte ATP release assay level; and
(C) comparing said treated erythrocyte ATP release assay level with control erythrocyte ATP release assay level, so as to determine the relative efficacy of said compound.

27. A method according to claim 26, for assessing the efficacy of treatment of a human or other animal subject having a glucose metabolism disorder, wherein said treatment comprises administering to said subject said compound and said sample of erythrocytes is obtained from said subject.

28. A method according to claim 26, for identifying a candidate erythrocyte ATP-release modulator.

29. A method for screening substances to identify a candidate erythrocyte ATP-release modulator, comprising
(A) providing a test substance and a sample of erythrocytes
(B) contacting a first portion of said sample of erythrocytes with said substance to prepare treated erythrocytes;
(C) assaying said treated erythrocytes for their level of ATP release to obtain a treated erythrocyte ATP release assay level;
(D) assaying a second portion of said sample of erythrocytes to obtain a control erythrocyte ATP release assay level; and
(E) comparing said treated erythrocyte ATP release assay level with said control erythrocyte ATP release assay level.

30. A method according to claim 29, wherein said second portion of said sample of erythrocytes is not treated with an erythrocyte ATP release modulator.

31. A method according to claim 30, said test substance is further evaluated for utility as an erythrocyte ATP release modulator if said treated erythrocyte ATP release assay level is significantly greater than said control erythrocyte ATP release assay level.

32. A method according to claim 29, comprising contacting said second portion of said sample with a known erythrocyte ATP release modulator prior to said step of assaying said second portion.

33. A method according to claim 29, wherein said sample of erythrocytes comprises erythrocytes obtained from a plurality of samples of erythrocytes having characterized ATP release characteristics, such that statistically meaningful comparison of said treated erythrocyte assay level and said control erythrocyte ATP release assay level may be made without concomitantly performing the steps of assaying said treated erythrocytes and assaying said second portion.

34. A method according to claim 33, wherein said control ATP release assay level is a reference standard level determined by repeating said assaying of said second portion on a plurality of second portions.

35. A method for assessing the level of erythrocyte response modulation activity of an erythrocyte ATP-release response modulator, comprising
(A) providing a substance having erythrocyte response modulation activity, and a sample of erythrocytes;
(B) contacting erythrocytes of the sample with the substance to prepare treated erythrocytes;
(C) assaying the treated erythrocytes for their level of ATP release upon physical deformation; and
(D) comparing that level to a level of ATP release determined under identical conditions for untreated erythrocytes;
wherein the difference in the levels of ATP release provides a determination of the degree level of erythrocyte response modulation activity.

36. A method for assessing the efficacy of a treatment for an erythrocyte-membrane-altering pathological condition in a subject, comprising assaying erythrocytes of the treated subject for their level of ATP release upon physical deformation, and comparing that level: (A) to a normal range of ATP release, determined under identical conditions for healthy individuals; or (B) to an abnormal level of ATP release found in the pathological condition, determined under identical conditions for the untreated subject or for untreated others exhibiting the pathological condition; or (C) to both.

37. A method according to claim 36, wherein a significant change in the subject's ATP release level, to or toward the normal range (A), indicates that the treatment has a significant efficacy.

38. A method according to claim 36, wherein the pathological condition is sickle cell anemia, malaria, thalassemia, anemia, a glucose processing disorder, chronic fatigue syndrome, or an obesity-related condition.

39. A method according to claim 38, wherein the pathological condition is a glucose processing disorder that is diabetes or metabolic syndrome.

40. An apparatus for measuring the level of erythrocyte ATP release by erythrocytes, comprising:
(A) a fluid flow conduit having a first region having a first cross-sectional area and an adjacent second region having a cross-sectional area that is less than said first cross-sectional area;
(B) a biocompatible pump in fluid communication with said fluid flow conduit, operable to pump fluid from said first region to said second region; and
(C) a photodetector in optical communication with said second region of said fluid flow conduit.

41. An apparatus according to claim 40, wherein said photodetector is operable to detect luminescence from luciferase.

42. An apparatus according to claim 40, further comprising a biocompatible chamber in fluid communication with said pump.

43. An apparatus for determining the level of erythrocyte ATP release of a test sample comprising erythrocytes, said apparatus comprising:
(A) a reservoir containing a supply of a cell-compatible, luciferin-containing solution;
(B) a biocompatible fluid flow conduit of approximately elliptical cross-section geometry and having, at a point along the fluid flow path, either (1) a stationery constriction of or deflection in the fluid flow conduit, or (2) a flexible wall of the fluid flow conduit to which pressure can be applied to form a constriction of or deflection in the fluid flow conduit; and
(C) a pump operative to distribute fluid along the fluid flow conduit; and
(D) a photodetector that is capable of detecting, and recording the amount of, light of about 560 nm when generated within the fluid flow conduit at or about said point(s) along the fluid flow path (B1 or B2);
whereby, upon introduction of said sample of erythrocytes and luciferase into the fluid flow conduit, operation of the apparatus can result in (1) generation of light of about 560 nm within the fluid flow conduit at or about said point(s) and (2) detection of light so generated, the detected amount of light thereby indicating the level of erythrocyte ATP release.

44. The apparatus according to claim 43, wherein the fluid flow channel has an internal diameter of about or at least 50 um, except for the point(s) along the fluid flow path (B1 or B2) at which a constriction or deflection is located or is formed, which has an internal diameter of about 1 to about 20 μm.

45. The apparatus according to claim 43, wherein the point(s) along the fluid flow path (B1 or B2) at which a constriction or deflection is located or is formed, has an internal diameter of about 1 to about 10 μm.
